(19) United States
(12) Patent Application Publication
(10) Pub. No.: US 2026/0212952 A1
(43) Pub. Date: Jul. 23, 2026

(54) COMPUTER-AIDED SYSTEM OF DESIGNING A COMBINATIONAL DRUG AND METHOD THEREOF

(71) Applicant: Praexisio Taiwan Inc., New Taipei City (TW)

(72) Inventor: Lee-Wei Yang, New Taipei City (TW)

(73) Assignee: Praexisio Taiwan Inc., New Taipei City (TW)

(21) Appl. No.: 19/453,744

(22) Filed: Jan. 20, 2026

Related U.S. Application Data
(60) Provisional application No. 63/747,388, filed on Jan. 21, 2025.

(51) Int. Cl. G16B 15/30; G16B 15/20; G16B 40/20

(52) U.S. Cl. CPC G16B 15/30; G16B 15/20; G16B 40/20

(57) ABSTRACT

A computer-aided system of designing a combinational drug includes a host. The host includes a memory module, a docking simulation module and a processing module. The memory module stores drug candidates and an assigned protein structure of a target protein. The docking simulation module executes the step of docking simulation, wherein each drug candidates has a first fragment and a second fragment. The processing module executes the step of selecting two drug candidates and the step of generating a new drug structure. In the step of selecting two drug candidates, a first drug is selected and has a best on-target ability on the target protein; and a second drug is selected and has a best systemic ability on the target protein. In the step of generating the new drug structure, the new drug structure includes the first fragment of the first drug and the second fragment of the second drug.

### CROSS-REFERENCE TO RELATED APPLICATION

This application claims the benefit of US provisional application serial No. 63/747,388, filed on January 21, 2025. The entirety of the above-mentioned patent application is hereby incorporated by reference herein and made a part of the specification.

### BACKGROUND

### TECHNICAL FIELD

Provided are a computer-aided system of designing a combinational drug and a method thereof.

### Related Art

Currently, the process of drug research and development roughly includes three stages, namely, Stage I "drug discovery", Stage II "preclinical development" and Stage III "clinical development". After completion of the three Stages, the applications of drug permit license will be applied in various countries. In Stage I "drug discovery", researchers need to obtain lead compounds based on studies of druggable sites in the target protein, and then proceed lead optimization of the lead compounds. For the lead optimization known to the inventor, virtual screening and molecular dynamics simulation screening may be applied in the process. According to the simulation results, researchers may carry out trials on drug candidates to test the effectiveness and cytotoxicity of the drug in Stage II and Stage III. However, even with the assistance of simulation tools, the researchers may obtain nearly tens of thousands of the drug candidates duo to lacking precision. Then, a lot of time and money is required to validate the drug candidates in Stage II and Stage III. Maybe, one drug candidate would be left for applying the drug permit license in the end. Thus, the cost of developing a new drug is always high.

### SUMMARY

In view of the foregoing issues, the instant disclosure provides a computer-aided system of designing a combinational drug and a method thereof.

According to one or some embodiments, the computer-aided method of designing a combinational drug is performed by a host, and the method comprises the following steps.

A step of docking simulation: in this step, according to drug candidates and an assigned protein structure of a target protein, docking the assigned protein structure for each of the drug candidates to obtain a plurality of complex structures during docking simulations. In each of the docking simulations, each of the drug candidates comprises a first fragment and a second fragment. The first fragment is defined as a fragment having an encountered relationship with an active site of the target protein. The second fragment is defined as a fragment not having the encountered relationship with the active site of the target protein. The encountered relationship is defined as a distance between a heavy atom of the active site of the target protein and a heavy atom of the drug candidate within a predetermined distance range.

A step of selecting a first drug according to a first criterion feature: in this step, the first drug is one of the drug candidates having a best on-target ability on the target protein.

A step of selecting a second drug according to a second criterion feature: in this step, the second drug is another of the drug candidates having a best systemic ability on the target protein.

A step of generating a new drug structure: in this step, the new drug structure comprises the first fragment of the first drug and the second fragment of the second drug.

Besides, according to one or some embodiments, the computer-aided method of designing a combinational drug is performed by a host, and the method comprises the following steps.

A step of docking simulation: in this step, according to drug candidates and an assigned protein structure of a target protein, docking the assigned protein structure for each of the drug candidates to obtain a plurality of complex structures during docking simulations. In each of the docking simulations, each of the drug candidates comprises a first fragment and a second fragment. The first fragment is defined as a fragment having an encountered relationship with an active site of the target protein. The second fragment is defined as a fragment not having the encountered relationship with the active site of the target protein. The encountered relationship is defined as a distance between a heavy atom of the active site of the target protein and a heavy atom of the drug candidate within a predetermined distance range.

A step of generating a first ranking list: in this step, according to a first criterion feature, ranking the drug candidates to generate the first ranking list.

A step of generating a second ranking list: in this step, according to a second criterion feature, ranking the drug candidates to generate the second ranking list.

A step of selecting a first drug: in this step, selecting one of the drug candidates as the first drug having a best rank of the first ranking list.

A step of selecting a second drug: in this step, selecting one of the drug candidates as the second drug having a best rank of the second ranking list.

A step of generating a new drug structure: in this step, the new drug structure comprises the first fragment of the first drug and the second fragment of the second drug.

In addition, according to one or some embodiments, the instant disclosure provides a computer-aided system of designing a combinational drug, including a host. The host includes a memory module, a docking simulation module and a processing module. The memory module stores an assigned protein structure of a target protein and a plurality of drug candidates. The docking simulation module is connected to the memory module and executes the step of docking simulation as described above. The processing module is connected to the memory module and the docking simulation module, and executes the steps of generating the first ranking list, generating the second ranking list, selecting the first drug, and selecting the second drug as described above.

Based on the above, according to one or some embodiments, the computer-aided system or method of designing a combinational drug is to apply molecular and non-molecular data feedbacks to design drugs. The designed combinational new drug has molecular level benefits and non-molecular level benefits. Therefore, for the drug research and development, the researchers can obtain the drug with good efficacy in the molecular level, and moreover, this drug also has good efficacy in the non-molecular as cell-based system level or animal-based system level. A success rate of trials can be improved, which is time-saving. In addition, the combinational new drug can be expected to have low toxicity and fewer side effects, so that the cost of developing a new drug can be reduced.

### BRIEF DESCRIPTION OF THE DRAWINGS

The instant disclosure will become more fully understood from the detailed description given herein below for illustration only, and thus not limitative of the instant disclosure, wherein:

FIG. 1 illustrates a block diagram of a computer-aided system of designing a combinational drug according to some embodiments;

FIG. 2 illustrates a flowchart (I) of a computer-aided method of designing a combinational drug according to some embodiments;

FIG. 3 illustrates a partial schematic diagram of a complex structure according to some embodiments, which shows an encountered relationship between a drug candidate and an assigned residue of a target protein, where areas surrounded by a dash dotted line represents a first fragment and a second fragment of the drug candidate;

FIG. 4 illustrates a schematic diagram of the computer-aided method of designing a combinational drug according to some embodiments, which shows that a generated new drug includes a first fragment of a drug candidate having the best activity inhibition rank and a second fragment of a drug candidate having the best growth inhibition rank;

FIG. 5 illustrates a flowchart of the step S11 of the computer-aided method of designing a combinational drug according to some embodiments;

FIG. 6 illustrates a partial schematic diagram of a pose according to some embodiments, which shows a distance between a center of mass of a drug candidate and a center of mass of an active site of an assigned protein structure;

FIG. 7 illustrates a flowchart (II) of a computer-aided method of designing a combinational drug according to some embodiments; and

FIG. 8 illustrates a flowchart (III) of a computer-aided method of designing a combinational drug according to some embodiments.

### DETAILED DESCRIPTION

In the following embodiments, the connection or coupling between units or modules refers to information transmission, which can be unidirectional or bidirectional, and the information transmission may be, for example, the reception or sending of messages or images, or the reception or sending of instructions, but is not limited thereto. The information transmission may include information transmission by direct electrical coupling, or by wireless communication technology such as Low Power Wide Area (LoRa), Bluetooth, WiFi or ZigBee, or by Internet such as a fixed line network, a coaxial cable, ADSL (Asymmetric Digital Subscriber Loop) or a mobile network (3G, 4G, 5G mobile Internet access), which are only embodiments but are not limited thereto.

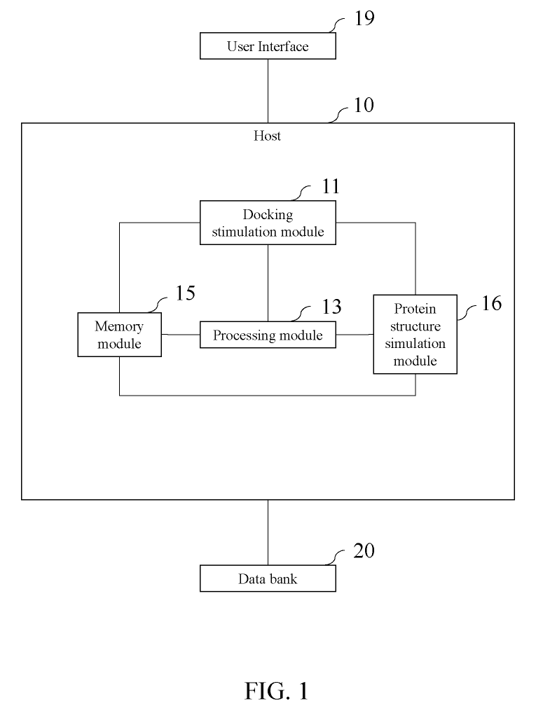

Please refer to FIG. 1. FIG. 1 illustrates a block diagram of a computer-aided system of designing a combinational drug according to some embodiments. The computer-aided system of designing a combinational drug includes a host 10. The host 10 may be, but not limited to, a computer or a cloud server. The host 10 includes a docking simulation module 11, a processing module 13 and a memory module 15. The memory module 15 stores drug candidates and an assigned protein structure of a target protein. The memory module 15 may be, but not limited to, various storage units, such as a hard disk, a solid-state drive (SSD) or various memory cards. The docking simulation module 11 is connected to the memory module 15 and the processing module 13. The docking simulation module 11 may be implemented by a processor in cooperation with docking simulation programs. The docking programs may be, but not limited to, AutoDock Vina, Dock or Glide. The processing module 13 is connected to the memory module 15. The processing module 13 may be implemented by a processor in cooperation with programs. It should be noted that the modules included in the host 10 are not limited to the docking simulation module 11, the processing module 13 and the memory module 15, and may also include embodiments of other modules (details of these embodiments will be described later).

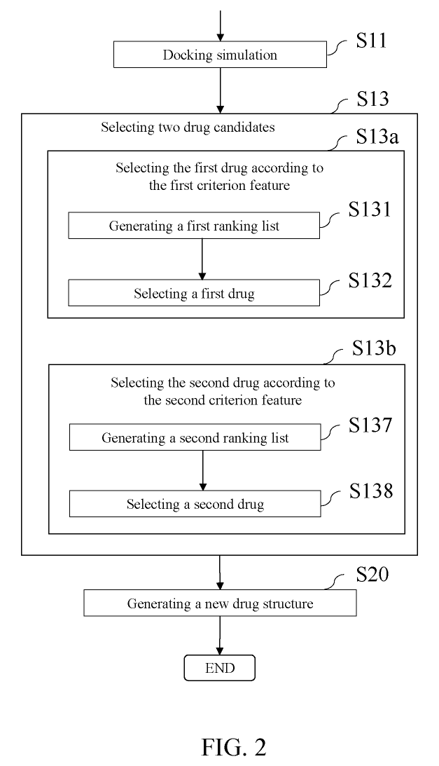

Please refer to FIG. 1 and FIG. 2. FIG. 2 illustrates a flowchart (I) of a computer-aided method of designing a combinational drug according to some embodiments. The computer-aided method of designing a combinational drug is performed by the host 10, and the method comprises the steps S11, S13 and S20. The step S11 is the step of docking simulation (details of the step S11 will be described later), and the docking simulation module 11 executes the step S11. The step S13 is the step of selecting two drug candidates and the step S20 is the step of generating a new drug structure (details of the steps S13 and S20 will be described later). The processing module 13 executes the steps S13 and S20.

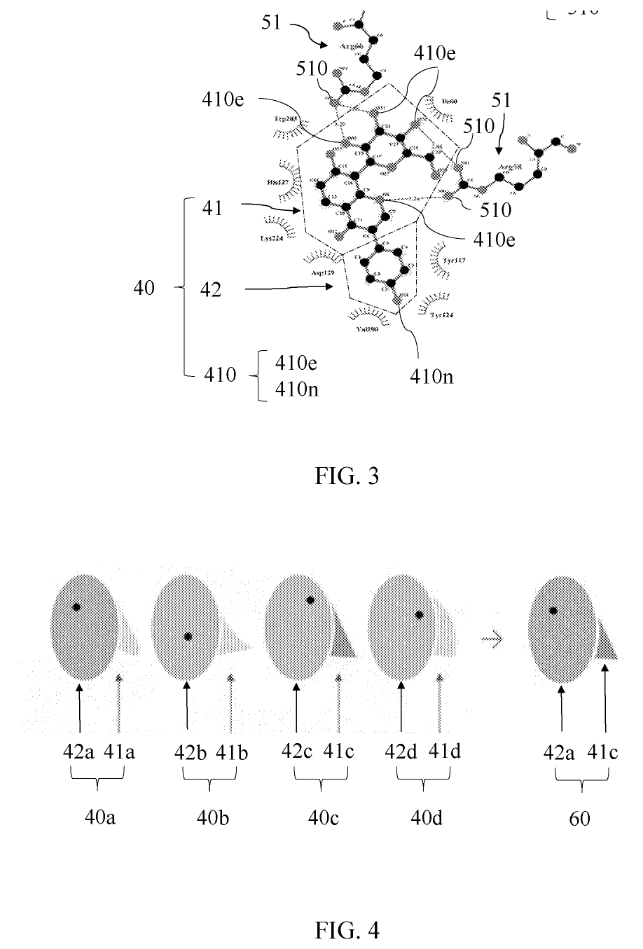

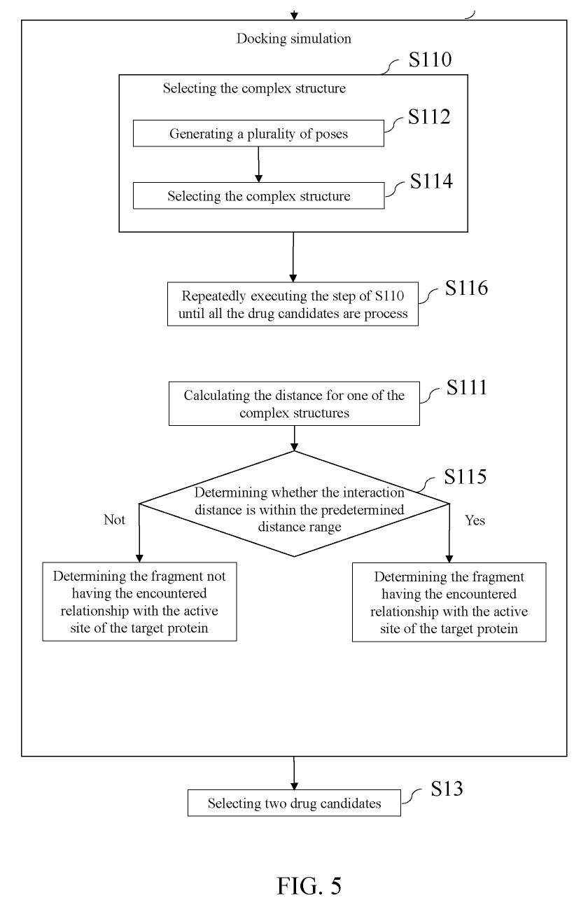

Please refer to FIG. 2, FIG. 3, FIG. 4 and FIG. 5. FIG. 3 illustrates a partial schematic diagram of a complex structure 30 according to some embodiments, which shows an encountered relationship between a drug candidate 40 and an assigned residue 51 of a target protein 50, where areas surrounded by a dash dotted line represents a first fragment 41 and a second fragment 42 of the drug candidate 40. FIG. 4 illustrates a schematic diagram of the computer-aided method of designing a combinational drug according to some embodiments, which shows that a generated new drug includes a first fragment 41 of a drug candidate 40 having the best activity inhibition rank and a second fragment 42 of a drug candidate 40 having the best growth inhibition rank. FIG. 5 illustrates a flowchart of the step S11 of the computer-aided method of designing a combinational drug according to some embodiments.

Please refer to FIG. 5. In the step S11, according to drug candidates and the assigned protein structure of the target protein, docking the assigned protein structure for each of the drug candidates to obtain a plurality of complex structures during docking simulations. In each of the docking simulations, each of the drug candidates comprises a first fragment and a second fragment. The first fragment is defined as a fragment having an encountered relationship with an active site of the target protein. The second fragment is defined as a fragment not having the encountered relationship with the active site of the target protein. The encountered relationship is defined as a distance between a heavy atom of the active site of the target protein and a heavy atom of the drug candidate within a predetermined distance range. In some embodiments, the encountered relationship is determined by the distance between the heavy atoms from the drug candidate and the active site of the target protein, respectively. In some embodiments, the encountered relationship may represent that the heavy atom of the drug candidate encounters the heavy atom of the active site of the target protein.

In some of embodiments, please refer to FIG. 3. The drug candidate 40 for the complex structure 30 includes an on-target fragment 41 (namely the first fragment) and a systemic fragment 42 (namely the second fragment). The on-target fragment 41 has the encountered relationship with the active site of the target protein 50, and the systemic fragment 42 does not have the encountered relationship with the active site of the target protein 50. The assigned residue 51 may be one of the residues at the active site of the target protein 50.

Please refer to FIG. 2. The step S13, specifically, includes the steps S13a and S13b. The step S13a is the step of selecting a first drug according to a first criterion feature. In the step S13a, the first drug is one of the drug candidates having a best on-target ability on the target protein. The step S13b is the step of selecting a second drug according to a second criterion feature. In the step S13b, the second drug is another of the drug candidates having a best systemic ability on the target protein. In some embodiments, the step S13 is to obtain two drug candidates, wherein one has the best on-target ability on the target protein, and the other one has the best systemic ability on the target protein. The step S20 is the step of generating a new drug structure. In the step S20, the new drug structure comprises the first fragment of the first drug and the second fragment of the second drug. In some embodiments, the “on-target ability on the target protein” may be defined as a binding affinity of the drug on an active site of the target protein. The better on-target ability, the more molecular interaction of the drug on the active site of the target protein. The molecular interaction may comprise the intermolecular interactions of a hydrogen bonding force, a van der Waals force, a salt bridge, or/and a secondary bond. In some embodiments, the drug with the better on-target ability may have the higher specificity on the target protein. In some embodiments, the “systemic ability on the target protein” may be defined as a binding affinity of the drug on an allosteric site of the target protein. The allosteric site is regulatory in function and it provides a binding site for the effectors that either activate or inhibit the target protein’s catalytic efficiency. In other words, the allosteric site and the active site may be two different sites of the target protein. In some embodiments, the combinational drug may be defined as a drug structure comprising two fragments from two different drugs, or a drug structure comprising two fragments from an active-site drug and an intermolecular allosteric drug.

Please refer to FIG. 2. In some embodiments, the steps S13a includes the steps S131 and S132. The step S131 is the step of generating a first ranking list. In the step S131, according to a first criterion feature, ranking the drug candidates to generate the first ranking list. The step S132 is the step of selecting a first drug. In the step S132, one of the drug candidates is selected as the first drug, and the first drug is the one having a best rank of the first ranking list. Further, the steps S13b includes the steps S137 and S138. The step S137 is the step of generating a second ranking list. In the step S137, according to a second criterion feature, ranking the drug candidates to generate the second ranking list. The step S138 is the step of selecting a second drug. In the step S138, one of the drug candidates is selected as the second drug, and the second drug is the one having a best rank of the second ranking list. In some embodiments, the step S13 is to obtain two drug candidates, wherein one has the best rank of the first ranking list, and the other one has the best rank of the second ranking list.

Therefore, the computer-aided system or method of designing a combinational drug is to apply molecular and non-molecular data feedbacks to generate a new drug structure for the combinational drug. The new drug structure including the effective on-target fragment and the effective systemic fragment from the two drugs respectively. Thus, the combinational new drug has molecular level benefits and non-molecular level benefits.

Please refer to FIG. 1, in some embodiments, the computer-aided drug design system further includes a user interface 19. The user interface 19 is connected to the host 10 and configured to receive the results from biochemical experiments or computer predictions as well as the results from cell, organism or animal experiments. The biochemical experiments may be, but not limited to, Co-Immunoprecipitation (Co-IP) experiments for protein-protein interactions, or an activity test method designed for enzyme molecules. The cell, organism or animal experiments are to detect the growth inhibition effect of the drug on the target cell, such as cytotoxicity tests or Xenograft Model animal experiments. The user interface 19 may be, but not limited to, a screen, a keyboard, a mouse, a touch screen or any combination of the foregoing units. In some embodiments, the memory module 15 may store the results from biochemical experiments or computer predictions as well as the results from cell, organism or animal experiments.

Please refer to FIG. 2, in some embodiments, in the step S13a, the first criterion feature comprises results from biochemical experiments or computer predictions. In the step S13b, the second criterion feature comprises results from cell, organism or animal experiments. In some embodiments, in the step S131, the first criterion feature represents a molecular level ability of each of the drug candidates binding to the target protein. In the step S137, the second criterion feature represents a non-molecular level ability of each of the drug candidates binding to the target protein. In some embodiments, the molecular level ability may be used to describe the on-target ability of the drug on the target protein. The non-molecular level ability may be used to describe the systemic ability of the drug on the target protein. In some embodiments, in the step S131, the first criterion feature is an activity inhibition feature comprising a half-maximal inhibitory concentration (IC₅₀) of each of the drug candidates. An activity inhibition ranking list is generated in the step S131. In the activity inhibition ranking list, one of the drug candidates with a lower IC₅₀ has a better rank than one with a higher IC₅₀. That is, the lower IC₅₀, the better rank of the activity inhibition ranking list. In the step S137, the second criterion feature is a growth inhibition feature comprising a half-maximal effective concentration (EC₅₀) of each of the drug candidates. A growth inhibition ranking list is generated in the step S137. In the growth inhibition ranking list, one of the drug candidates with a lower EC₅₀ has a better rank than one with a higher EC₅₀. That is, the lower EC₅₀, the better rank of the growth inhibition ranking list.

Please refer to FIG. 5. In some embodiments, in the step S11, one of the complex structures is obtained in one of the docking simulations. The step S11 includes the steps S111 and S115. The step S111 is the step of calculating the distance for one of the complex structures, wherein the distance is the distance between the heavy atom of the active site of the target protein and the heavy atom of the drug candidate. The step S115 is the step of determining whether the distance is within the predetermined distance range. If yes, determining the fragment having the encountered relationship with the active site of the target protein; if not, determining the fragment not having the encountered relationship with the active site of the target protein. The steps S111 and S115 are repeatedly executed until all the complex structures are processed. In some embodiments, the first fragment comprises a functional group encountering the active site of the target protein. The second fragment comprises a functional group not encountering the active site of the target protein.

Please refer to FIG. 3, for example, in the step S11, for the complex structure 30, the distance between the heavy atom 410 of the drug candidate 40 and the heavy atom 510 of the assigned residue 51 at the active site of the target protein 50 is calculated. If the distance is within the predetermined distance range, it may represent that the heavy atom 410 of the drug candidate 40 is the heavy atom 410e encountering the heavy atom 510 of the assigned residue 51. The predetermined distance range may be, but not be limited to, within a range of 0 angstroms (Å) to 5 angstroms (Å), preferably 3 angstroms (Å) to 5 angstroms (Å). If the distance is greater than the predetermined distance range, it may represent that the heavy atom 410 of the drug candidate 40 is the heavy atom 410n that do not encounter the heavy atom 510 of the assigned residue 51. Further, according to positions of the heavy atoms 410e and the heavy atoms 410n in the structure of the drug candidate 40, the fragments of the structure are respectively defined as the on-target fragment 41 and the systemic fragment 42. Since each of the drug candidates 40 is processed in the step S11, each of the drug candidates 40 includes two fragments, wherein one encounters the active site of the target protein 50 and the other does not.

Please refer to FIG. 4. In some embodiments, the drug candidate 40 has an on-target fragment 41 and a systemic fragment 42. For example, there are four drug candidates 40 are processed in the step S11. Each of the four drug candidates 40a, 40b, 40c, 40d has the on-target fragments 41a, 41b, 41c, 41d and the systemic fragments 42a, 42b, 42c, 42d, respectively. In the step S131, the four drug candidates 40a, 40b, 40c, 40d are ranked according to the activity inhibition feature, and the activity inhibition ranking list is generated. In the order of ranks, the drug candidate 40c is the best, followed by the drug candidate 40b and the drug candidate 40a, and the drug candidate 40d is the worst. In the step S132, the drug candidate 40c is selected as the first drug. The drug candidate 40c has the best on-target ability on the target protein 50, or the best molecular level ability of binding to the target protein 50. In other words, the drug candidate 40c has the strongest affinity for the target protein and can effectively inhibit the activity of the target protein. In the step S137, the four drug candidates 40a, 40b, 40c, 40d are ranked according to the growth inhibition feature, and the growth inhibition ranking list is generated. In the order of ranks, the drug candidate 40a is the best, followed by the drug candidate 40b and the drug candidate 40c, and the drug candidate 40d is the worst. In the step S138, the drug candidate 40a is selected as the second drug. The drug candidate 40a has the best systemic ability on the target protein 50, or the best non-molecular level ability of binding to the target protein 50. In other words, the drug candidate 40a is highly toxic to the target cell and its efficacy of inhibiting the growth of the target cell is the best. As the result, in the step 20, the structure of the new drug 60 is generated, and includes the on-target fragment 41c of the drug candidate 40c and the systemic fragment 42a of the drug candidate 40a, as shown in FIG. 4.

Please refer to FIG. 3. In some embodiments, the on-target fragment 41 includes at least one functional group, which has the encountered relationship with the active site of the target protein 50. That is, the functional group of the on-target fragment 41 has the heavy atom 410e, which encountering the heavy atom 510 of the assigned residue 51. In some embodiments, the systemic fragment 42 includes at least one functional group not encountering the active site of the target protein 50. That is, the functional group of the systemic fragment 42 has the heavy atom 410n, which has no encountered relationship with the heavy atom 510 of the assigned residue 51. The functional group may be, but not limited to, an amino group (-NH₂), a carboxyl group (-COOH), an acyl group or amide. In some embodiments, the heavy atoms 410 and 510 are non-hydrogen atoms, for example, nitrogen atoms or oxygen atoms. In some embodiments, the heavy atoms 410 and 510 may be atoms with the electronegativity greater than the electronegativity of hydrogen atom. In some embodiments, the heavy atoms 410 and 510 may be non-hydrogen atoms with the electronegativity greater than the electronegativity of carbon atom.

In some embodiments, the target cell may be cancer cells (such as breast cancer cells). The target protein is a growth promoting factor of the target cell, such as EgIN2, an inducible estrogen in breast carcinoma cells.

Please refer to FIG. 5. In some embodiments, the step S110, which is to select the complex structure. The step S11 further includes the steps S110 and S116. The step S110 is the step of selecting the complex structure in one of the docking simulations. The step S110 includes the steps S112 and S114. The step S112 is the step of docking the assigned structure with the drug candidate to generate a plurality of poses. The step S114 is the step of selecting one of the poses as the complex structure having a smallest mass distance. The mass distance is a distance between the center of mass of the active site of the assigned protein structure and the center of mass of the drug candidate. The step S116 is the step of repeatedly executing the step S110 until all the drug candidates are processed.

In some embodiments, the steps S110, S112, S114 and S116 are performed during molecular dynamic simulations. The molecular dynamic simulations for the poses were performed by the host 10 (or the docking simulation module 11) in FIG. 1 using the OpenMM package in an explicit solvent. In some embodiments, the simulation model of the aforesaid poses is prepared by using the LEaP program in AmberTools. The complex of target protein-drug binding pose was solvated using an explicit solvent of the TIP3P water model, with at least 10 Å of water layer patched on each side of the water box between the protein target and the box boundary. Sodium and chloride ions were used to neutralize the system to achieve a salt concentration of 100 mM. The system was first energy minimized for all the hydrogen, waters, and ions positions, leaving the remaining atoms restrained using a force constant of 10 kcal/mol/Å².

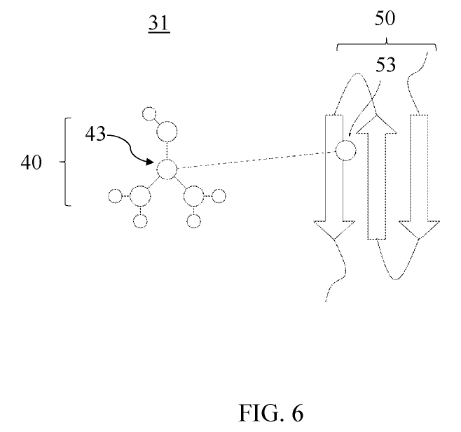

Please refer to FIG. 6. FIG. 6 illustrates a partial schematic diagram of a pose 31 according to some embodiments, which shows a distance between a center of mass 43 of a drug candidate and a center of mass 53 of an active site of an assigned protein structure. For example, in the step S110, the mass distance of each pose 31 is calculated. The pose 31 with the smallest mass distance is selected. In FIG. 6, for example, the pose 31 is selected as the complex structure 30, because in the pose 31, the center of mass 43 of the drug candidate 40 is closest to the center of mass 53 of the active site of the target protein 50.

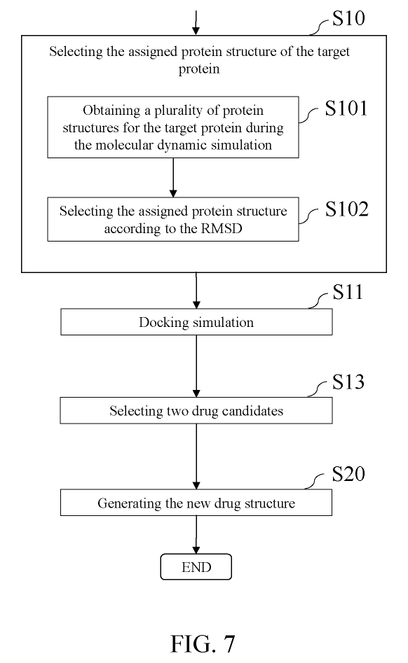

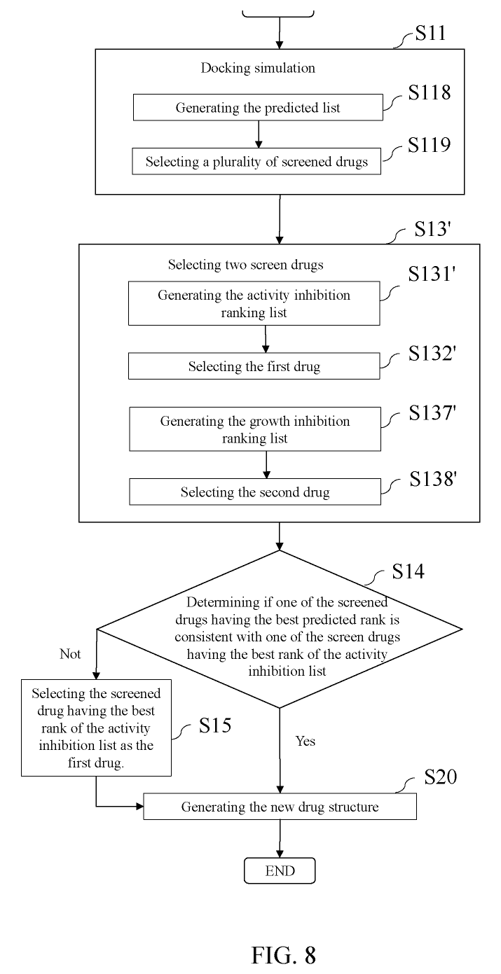

Please refer to FIG. 1 and FIG. 7. FIG. 7 illustrates a flowchart (II) of a computer-aided method of designing a combinational drug according to some embodiments. In some embodiments, the host 10 further includes a protein structure simulation module 16. The protein structure simulation module 16 is connected to the processing module 13 and the docking simulation module 11. The protein structure simulation module 16 executes the step S10. The step S10 is the step of selecting the assigned protein structure (details of the step S10 will be described later). The protein structure simulation module 16 may be implemented by processors in cooperation with programs. In some embodiments, the host 10 is connected to the data bank 20 to obtain a plurality of protein structures of the target protein. The data bank 20 may be, but not limited to, Protein Data Bank (PDB), GenBank, and SWISS-PROT. According to the sequence data from the data bank 20, the protein structure simulation module 16 may obtain the plurality of protein structures during simulations, and select one of them as the assigned protein structure. In some embodiments, the operator operates in a drug screening webpage displayed by the user interface 19 to receive the assigned structure of the target protein and the plurality of drug candidates.

Please refer to FIG. 7. In some embodiments, the step S10 includes the steps S101 and S102. The step S101 is the step of obtaining protein structures for the target protein during a molecular dynamic simulation. The step S102 is the step of selecting one of the protein structures as the assigned protein structure having a lowest root-mean-square deviation (RMSD) within a predetermined time. That is, the step S102 is selecting the assigned protein structure according to the RMSD of each of the protein structures, and the assigned protein structure has the lowest RMSD within the predetermined time. For example, in molecular dynamic simulations, all simulated structures presented by the target protein within 100 nanoseconds (ns) are taken, and the simulated structure having the lowest root-mean-square deviation is selected as the assigned protein structure. The assigned protein structure is the simulated structure maintained for the longest time within this predetermined time 100 ns. Therefore, the assigned protein structure is the most stable protein structure. The simulation result in the step S11 subsequently can be closer to the true binding pose of the drug and the target protein in living cells, thereby reducing the error of the simulation result.

Please refer to FIG. 8. FIG. 8 illustrates a flowchart (III) of a computer-aided method of designing a combinational drug according to some embodiments. In some embodiments, the step S11 further includes the steps S118 and S119. The step S118 is the step of generate a predicted list. In the step S118, according to a free energy of each of the complex structures, the drug candidates are ranked to generate the predicted list. The step S119 is the step of selecting a plurality of screened drugs from the drug candidates of the predicted list. Each of the screened drugs has a predicted rank, and their predicted ranks are within a predetermined ranking range. In other words, in the predicted list, the drug candidates in the top rank (for example, the candidates in the top four) are selected as the screened drugs. In this embodiment, the drug candidates can be screened first in the simulations, and the screened drugs may be subjected to the subsequent steps S13’, S14, S15, and S20.

In the step S13’, the screened drugs are subjected to be selected. In some embodiments, the step S131’ is the step of ranking the screened drugs according to the activity inhibition feature of each of the screened drugs on the target protein, and generating the activity inhibition ranking list. The step S132' is the step of selecting the first drug which is one of the screened drugs having the best rank of the activity inhibition ranking list. The step S137' is the step of ranking the screened drugs according to the growth inhibition feature of each of the screened drugs on the target cell, and generating the growth inhibition ranking list. The step 138' is the step of selecting the second drug which is one of the screened drugs having the best rank of the best growth inhibition ranking list.

In some embodiments, the processing module 13 executes the steps S14 and S15. The step S14 is the step of determining if one of the screened drugs having the best predicted rank is consistent with one of the screened drugs having the best rank of the first ranking list. If yes, the processing module 13 executes the step S20; if not, the processing module 13 executes the steps S15 and S20. The step S15 is the step of selecting the screened drug having the best rank of the first ranking list as the first drug.

In some embodiments, the host 10 is connected to the data bank 20 to obtain data of a plurality of approved drugs. The data bank 20 may be, but not limited to, a MedChemExpress (MCE) FDA approved drug data bank (Cat. No.:HY-L022), or a Screen Well^{®}FDA approved drug data bank (version 1.5) of Enzo Life Sciences. Then, the docking simulation module 11 executes drug screening on the drugs to obtain the plurality of drug candidates. In some embodiments, the host 10 is connected to a drug screening platform (not shown). The drug screening platform has the function of the drug screening module, and executes drug screening to obtain a plurality of drug candidates. The host 10 receives the drug candidates, and the processing module 13 executes the computer-aided method of designing a combinational drug on the drug candidates. In some embodiments, the drug candidates are approved small molecule drugs.

In some embodiments, in the step S118, the free energy approximated by the enthalpy contribution between the target protein and drugs for each sampled snapshot was calculated by MM/GBSA methods with MMPBSA.py module in the AmberTools. The simulation results were summarized using the designed indicators, including the mean binding free energy from MM/GBSA calculation over sampled snapshots, the drug leaving time when the drug center of mass moving away from the target site more than 10 Å, and the largest distance of the drug COM to the target sites sampled during the simulations.

Based on the above, according to one or some embodiments, the computer-aided system or method of designing a combinational drug is to apply molecular and non-molecular data feedbacks to design drugs. The designed combinational new drug has molecular level benefits and non-molecular level benefits. Therefore, for the drug research and development, the researchers can obtain the drug with good efficacy in the molecular level, and moreover, this drug also has good efficacy in the non-molecular as cell-based system level or animal-based system level. A success rate of trials can be improved, which is time-saving. In addition, the combinational new drug can be expected to have low toxicity and fewer side effects, so that the cost of developing a new drug can be reduced.

While the instant disclosure has been described by the way of example and in terms of the preferred embodiments, it is to be understood that the invention need not be limited to the disclosed embodiments. On the contrary, it is intended to cover various modifications and similar arrangements included within the spirit and scope of the appended claims, the scope of which should be accorded the broadest interpretation so as to encompass all such modifications and similar structures.

What is claimed is:

1. A computer-aided method of designing a combinational drug, performed by a host, the method comprising:
according to drug candidates and an assigned protein structure of a target protein, docking the assigned protein structure for each of the drug candidates to obtain a plurality of complex structures during docking simulations, wherein in each of the docking simulations, each of the drug candidates comprises a first fragment and a second fragment; the first fragment is defined as a fragment having an encountered relationship with an active site of the target protein; the second fragment is defined as a fragment not having the encountered relationship with the active site of the target protein; and the encountered relationship is defined as a distance between a heavy atom of the active site of the target protein and a heavy atom of the drug candidate within a predetermined distance range;
selecting a first drug according to a first criterion feature, wherein the first drug is one of the drug candidates having a best on-target ability on the target protein;
selecting a second drug according to a second criterion feature, wherein the second drug is another of the drug candidates having a best systemic ability on the target protein; and
generating a new drug structure, the new drug structure comprising the first fragment of the first drug and the second fragment of the second drug.

2. The computer-aided method of designing a combinational drug according to claim 1, wherein the first criterion feature comprises results from biochemical experiments or computer predictions; and the second criterion feature comprises results from cell, organism or animal experiments.

3. The computer-aided method of designing a combinational drug according to claim 1, wherein the step of selecting the first drug according to the first criterion feature comprises:
according to the first criterion feature, ranking the drug candidates to generate a first ranking list; and
selecting one of the drug candidates as the first drug having a best rank of the first ranking list; and
the step of selecting the second drug according to the second criterion feature comprises:
according to the second criterion feature, ranking the drug candidates to generate a second ranking list; and
selecting one of the drug candidates as the second drug having a best rank of the second ranking list.

4. The computer-aided method of designing a combinational drug according to claim 3, wherein the first criterion feature is an activity inhibition feature comprising a half-maximal inhibitory concentration (IC₅₀) of each of the drug candidates; the second criterion feature is a growth inhibition feature comprising a half-maximal effective concentration (EC₅₀) of each of the drug candidates;
in the step of generating the first ranking list, one of the drug candidates with a lower IC₅₀ has a better rank than one with a higher IC₅₀ in the first ranking list; and
in the step of generating the second ranking list, one of the drug candidates with a lower EC₅₀ has a better rank than one with a higher EC₅₀ in the second ranking list.

5. The computer-aided method of designing a combinational drug according to claim 3, the step of the docking simulations further comprising:
according to a molecular-dynamic simulation based free energy calculation of each of the complex structures, ranking the drug candidates to generate a predicted list; and
selecting screened drugs from the predicted list, wherein each of the screened drugs has a predicted rank within a predetermined ranking range; in the step of generating the first ranking list, ranking the screened drugs to generate the first ranking list; in the step of selecting the first drug, the first drug is the selected screened drug; in the step of generating the second ranking list, ranking the screened drugs to generate the second ranking list; in the step of selecting the second drug, the second drug is the selected screened drug; and
before the step of generating the new drug structure, the method further comprises:
determining if one of the screened drugs having the best predicted rank is consistent with one of the screened drugs having the best rank of the first ranking list, wherein if not, selecting the screened drug having the best rank of the first ranking list as the first drug.

6. The computer-aided method of designing a combinational drug according to claim 1, wherein in the step of the docking simulations, one of the complex structures is obtained in one of the docking simulations; the step of the docking simulations comprises:
calculating the distance for one of the complex structures;
determining whether the distance is within the predetermined distance range, wherein if yes, determining the fragment having the encountered relationship with the active site of the target protein; if not, determining the fragment not having the encountered relationship with the active site of the target protein;
selecting the complex structure in one of the docking simulations, comprising:
docking the assigned protein structure with one of the drug candidates to generate poses; and
selecting one of the poses as the complex structure having a smallest mass distance; and
repeatedly executing the step of selecting the complex structure until all the drug candidates are processed.

7. The computer-aided method of designing a combinational drug according to claim 1, wherein before the step of the docking simulations, the method further comprises:
selecting the assigned protein structure, comprising:
obtaining protein structures for the target protein during a molecular dynamic simulation; and
selecting one of the protein structures as the assigned protein structure having a lowest root-mean-square deviation (RMSD) within a predetermined time.

8. A computer-aided method of designing a combinational drug, performed by a host, the method comprising:
according to drug candidates and an assigned protein structure of a target protein, docking the assigned protein structure for each of the drug candidates to obtain a plurality of complex structures during docking simulations, wherein in the docking simulation, each of the drug candidates comprises a first fragment and a second fragment; the first fragment is defined as a fragment having an encountered relationship with an active site of the target protein; the second fragment is defined as a fragment not having the encountered relationship with the active site of the target protein; and the encountered relationship is defined as a distance between a heavy atom of the active site of the target protein and a heavy atom of the drug candidate within a predetermined distance range;
according to a first criterion feature, ranking the drug candidates to generate a first ranking list;
according to a second criterion feature, ranking the drug candidates to generate a second ranking list;
selecting one of the drug candidates as a first drug having a best rank of the first ranking list;
selecting one of the drug candidates as a second drug having a best rank of the second ranking list; and
generating a new drug structure, the new drug structure comprising the first fragment of the first drug and the second fragment of the second drug.

9. The computer-aided method of designing a combinational drug according to claim 8, wherein the first criterion feature comprises results from biochemical experiments or computer predictions; and the second criterion feature comprises results from cell, organism or animal experiments.

10. The computer-aided method of designing a combinational drug according to claim 8, wherein the first criterion feature represents a molecular level ability of each of the drug candidates binding to the target protein; and the second criterion feature represents a non-molecular level ability of each of the drug candidates binding to the target protein.

11. The computer-aided method of designing a combinational drug according to claim 8, wherein the first criterion feature is an activity inhibition feature comprising a half-maximal inhibitory concentration (IC₅₀) of each of the drug candidates; the second criterion feature is a growth inhibition feature comprising a half-maximal effective concentration (EC₅₀) of each of the drug candidates;
in the step of generating the first ranking list, one of the drug candidates with a lower IC₅₀ has a better rank than one with a higher IC₅₀ in the first ranking list; and
in the step of generating the second ranking list, one of the drug candidates with a lower EC₅₀ has a better rank than one with a higher EC₅₀ in the second ranking list.

12. The computer-aided method of designing a combinational drug according to claim 8, the step of the docking simulations further comprising:
according to a molecular-dynamic simulation based free energy calculation of each of the complex structures, ranking the drug candidates to generate a predicted list; and
selecting screened drugs from the predicted list, wherein each of the screened drugs has a predicted rank within a predetermined ranking range; in the step of generating the first ranking list, ranking the screened drugs to generate the first ranking list; in the step of selecting the first drug, the first drug is the selected screened drug; in the step of generating the second ranking list, ranking the screened drugs to generate the second ranking list; in the step of selecting the second drug, the second drug is the selected screened drug; and
before the step of generating the new drug structure, the method further comprises:
determining if one of the screened drugs having the best predicted rank is consistent with one of the screened drugs having the best rank of the first ranking list, wherein if not, selecting the screened drug having the best rank of the first ranking list as the first drug.

13. The computer-aided method of designing a combinational drug according to claim 8, wherein in the step of the docking simulations, one of the complex structures is obtained in one of the docking simulations; the step of the docking simulations comprises:
calculating the distance for one of the complex structures;
determining whether the distance is within the predetermined distance range, wherein if yes, determining the fragment having the encountered relationship with the active site of the target protein; if not, determining the fragment not having the encountered relationship with the active site of the target protein;
selecting the complex structure in one of the docking simulations, comprising:
docking the assigned protein structure with one of the drug candidates to generate poses; and
selecting one of the poses as the complex structure having a smallest mass distance; and
repeatedly executing the step of selecting the complex structure until all the drug candidates are processed.

14. The computer-aided method of designing a combinational drug according to claim 8, wherein before the step of the docking simulations, the method further comprises:
selecting the assigned protein structure, comprising:
obtaining protein structures for the target protein during a molecular dynamic simulation; and
selecting one of the protein structures as the assigned protein structure having a lowest root-mean-square deviation (RMSD) within a predetermined time.

15. A computer-aided system of designing a combinational drug, comprising a host, the host comprising:
a memory module, storing drug candidates and an assigned protein structure of a target protein; and
a docking simulation module, connected to the memory module and executing the following steps:
according to drug candidates and an assigned protein structure of a target protein, docking the assigned protein structure for each of the drug candidates to obtain a plurality of complex structures during docking simulations, wherein in the docking simulation, each of the drug candidates comprises a first fragment and a second fragment; the first fragment is defined as a fragment having an encountered relationship with an active site of the target protein; the second fragment is defined as a fragment not having the encountered relationship with the active site of the target protein; and the encountered relationship is defined as a distance between a heavy atom of the active site of the target protein and a heavy atom of the drug candidate within a predetermined distance range;
a processing module, connected to the memory module and the docking simulation module and executing the following steps:
according to a first criterion feature, ranking the drug candidates to generate a first ranking list;
according to a second criterion feature, ranking the drug candidates to generate a second ranking list;
selecting one of the drug candidates as a first drug having a best rank of the first ranking list;
selecting one of the drug candidates as a second drug having a best rank of the second ranking list; and
generating a new drug structure, the new drug structure comprising the first fragment of the first drug and the second fragment of the second drug.

16. The computer-aided system of designing a combinational drug according to claim 15, further comprising:
a user interface connected to the host and receiving the results from biochemical experiments or computer predictions and the results from cell, organism or animal experiments, wherein the first criterion feature comprises results from biochemical experiments or computer predictions; and the second criterion feature comprises results from cell, organism or animal experiments.

17. The computer-aided system of designing a combinational drug according to claim 15, wherein the first criterion feature is an activity inhibition feature comprising a half-maximal inhibitory concentration (IC₅₀) of each of the drug candidates; the second criterion feature is a growth inhibition feature comprising a half-maximal effective concentration (EC₅₀) of each of the drug candidates;
in the first ranking list, one of the drug candidates with a lower IC₅₀ has a better rank than one with a higher IC₅₀; and
in the second ranking list, one of the drug candidates with a lower EC₅₀ has a better rank than one with a higher EC₅₀.

18. The computer-aided system of designing a combinational drug according to claim 15, wherein the docking simulation module executes the following steps:
according to a molecular-dynamic simulation based free energy calculation of each of the complex structures, ranking the drug candidates to generate a predicted list; and
selecting screened drugs from the predicted list, wherein each of the screened drugs has a predicted rank within a predetermined ranking range; in the step of generating the first ranking list, ranking the screened drugs to generate the first ranking list; in the step of selecting the first drug, the first drug is the selected screened drug; in the step of generating the second ranking list, ranking the screened drugs to generate the second ranking list; in the step of selecting the second drug, the second drug is the selected screened drug; and
the processing module executes the following steps:
determining if one of the screened drugs having the best predicted rank is consistent with one of the screened drugs having the best rank of the first ranking list, wherein if not, selecting the screened drug having the best rank of the first ranking list as the first drug.

19. The computer-aided system of designing a combinational drug according to claim 15, wherein the docking simulation module obtains one of the complex structures in one of the docking simulations and executes the following steps:
calculating the distance for one of the complex structures;
determining whether the distance is within the predetermined distance range, wherein if yes, determining the fragment having the encountered relationship with the active site of the target protein; if not, determining the fragment not having the encountered relationship with the active site of the target protein;
selecting the complex structure in one of the docking simulations, comprising:
docking the assigned protein structure with one of the drug candidates to generate poses; and
selecting one of the poses as the complex structure having a smallest mass distance; and
repeatedly executing the step of selecting the complex structure until all the drug candidates are processed.

20. The computer-aided system of designing a combinational drug according to claim 15, wherein the host further comprises:
a protein structure simulation module, connected to the processing module and the docking simulation module, and executing the following steps:
selecting the assigned protein structure, comprising:
obtaining protein structures for the target protein during a molecular dynamic simulation; and
selecting one of the protein structures as the assigned protein structure having a lowest root-mean-square deviation (RMSD) within a predetermined time.
